(19) United States
(12) Patent Application Publication
(10) Pub. No.: US 2005/0152868 A1
(43) Pub. Date: Jul. 14, 2005

(54) Deodorizing agent containing dry cell of microorganism as active ingredient and deodorizing method

(75) Inventors: Makoto Ebisawa, Saga-gun (JP); Yasuhiko Toride, Kawasaki-shi (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: AJINOMOTO CO., INC., Tokyo (JP)

(21) Appl. No.: 11/031,090

(22) Filed: Jan. 6, 2005

(30) Foreign Application Priority Data
Jan. 9, 2004 (JP) 4839/2004

(51) Int. Cl. A61L 9/00; A61L 9/01

(52) U.S. Cl. 424/76.1; 424/780

(57) ABSTRACT

The present invention provides a deodorizing agent and a deodorizing method capable of effectively deodorizing an offensive odor substance caused by, for example, animal excreta. The deodorizing agent and method utilize a dry cell of a microorganism selected from the group consisting of bacteria belonging to the genus Escherichia, bacteria belonging to the genus Brevibacterium, and bacteria belonging to the genus Bacillus, as an active ingredient.

### TECHNICAL FIELD

The present invention relates to a deodorizing agent comprising a dry cell of a microorganism as an active ingredient, which has a deodorizing effect on an odor substance, particularly, ammonia, trimethylamine, methyl mercaptan, hydrogen sulfide, fatty acid including propionic acid and n-butyric acid and the like. The present invention also relates to a deodorizing method comprising contacting the deodorizing agent with an odor substance.

### BACKGROUND ART

Conventionally, various offensive odor sources have been deodorized by chemical treatment methods using a deodorizing agent, physical treatment methods comprising use of an absorbent or burning an offensive odor substance (e.g., JP-A-2001-519, JP-A-2001-522, JP-A-2001-157706), and biological treatment methods comprising degrading an offensive odor substance with a microorganism and the like (e.g., JP-B-2810308).

The principle of a microbiological deodorizing method which decomposes an odor component using a particular group of microorganisms is decomposition by microorganisms of an odor substance that becomes the source of an offensive odor. In this method, the microorganism that decomposes an odor substance is a viable cell, and the odor substance is decomposed by the metabolic action of the viable cell.

In the method of physically adsorbing an odor component with an absorbent such as activated carbon and the like, the principle is adsorption of an odor component within the micropores of the absorbent. However, the adsorption ability per unit weight varies, and an odor component once adsorbed may be problematically desorbed.

In addition, zeolites and the like used for deodorizing cannot be reused and are difficult to be treated, posing a serious environmental problem as evidenced by the pressing need for final disposal sites and the like.

### DISCLOSURE OF THE INVENTION

Offensive odors caused by excreta etc. produced during the breeding of domestic animals and indoor breeding of pet animals such as dogs, cats, and the like have become a big problem in recent years. An object of the present invention is to provide a deodorizing agent capable of deodorizing an offensive odor, particularly a typical odor substance from the aforementioned excreta such as ammonia, trimethylamine, methyl mercaptan, hydrogen sulfide, fatty acid including propionic acid and n-butyric acid and the like, and a deodorizing method using the deodorizing agent.

The present inventors have found that dry cells of various bacteria (e.g., bacteria belonging to the genus Escherichia, bacteria belonging to the genus Brevibacterium, bacteria belonging to the genus Bacillus and the like) widely used for the industrial production of amino acids, nucleic acids, and the like can effectively eliminate odor substances such as ammonia, trimethylamine, methyl mercaptan, hydrogen sulfide, fatty acid including propionic acid and n-butyric acid and the like, by acting on an offensive odor substance, which resulted in the completion of the present invention.

That is, the present invention provides a deodorizing agent comprising a dry cell of a microorganism selected from the group consisting of bacteria belonging to the genus Escherichia, bacteria belonging to the genus Brevibacterium and bacteria belonging to the genus Bacillus as an active ingredient, and particularly the above-mentioned deodorizing agent wherein the deodorizing agent deals with at least one kind of odor substance selected from the group consisting of ammonia, trimethylamine, methyl mercaptan, hydrogen sulfide, and fatty acid including propionic acid and n-butyric acid.

The present invention also provides a deodorizing method comprising contacting the above-mentioned deodorizing agent with an odor substance.

### DETAILED DESCRIPTION OF THE INVENTION

The offensive odor substance as referred to in the present invention is free of any particular limitation and is typically an odor substance from excreta-of pet animals such as a dog, cat and the like due to indoor breeding or excreta when domestic animals such as cattle, pigs, chicken and the like are bred. More specifically, it is an odor substance of ammonia, hydrogen sulfide, trimethylamine, acetaldehyde, methyl mercaptan, fatty acid including propionic acid, n-butyric acid, n-valeric acid, iso-valeric acid and the like.

The bacteria belonging to the genus Escherichia, bacteria belonging to the genus Brevibacterium and bacteria belonging to the genus Bacillus to be used in the present invention are bacteria for amino acid fermentation or nucleic acid fermentation, and have an ability to produce amino acids or nucleic acids when sugar sources such as starch, molasses and the like are used as main starting materials, various nutrients necessary for the growth of useful microorganisms are added, and the microorganism is placed under optimal pH, temperature and aeration management. Preferably, cells after various fermentations are used.

As regards the amino acid fermentation microorganism, the amino acid produced by the fermentation microorganisms can be, for example, glutamic acid, lysine, arginine, phenylalanine, a branched chain amino acid (e.g., valine, leucine, isoleucine) and the like, with particular preference given to lysine, arginine and phenylalanine fermentation microorganisms. As regards the nucleic acid fermentation microorganism, 5′-inosinic acid, guanosine and inosine fermentation microorganisms are preferable. To be more specific, the fermentation microorganism of glutamic acid can be, for example, Brevibacterium flavum ATCC 14067. The fermentation microorganism of lysine can be, for example, Escherichia coli FERM BP-5252 and Brevibacterium flavum ATCC 21475. The fermentation microorganism of arginine can be, for example, Brevibacterium flavum FERM BP-6894. The fermentation microorganism of phenylalanine can be, for example, Escherichia coli FERM BP-3853, Brevibacterium lactofermentum FERM BP-4160 and Bacillus subtilis FERM BP-609. The fermentation microorganism of valine can be, for example, Brevibacterium lactofermentum FERM BP-1763.

In the present invention, a dry cell means a dead cell substantially free of water, which sometimes contains residual medium components, filter aids and the like. By the phrase “substantially free of water” is meant that the water content is not more than 15 wt %, preferably not more than 10 wt %.

The dry cell can be produced by any suitable technique but is generally produced in the following manner.

According to a known method, a cell is cultured in a culture medium managed to have pH, temperature and aeration suitable for each fermentation. Upon completion of the fermentation, the cell, which can be sterilized with heating or not sterilized, is collected by centrifugation or filtration.

The obtained cell, either as is or, for example, re-suspended in water, physiological saline, a suitable buffer near neutral and the like, is then subjected to the subsequent drying step.

The cell may be dried by any method, and, for example, industrially known spray drying, ventilation drying, lyophilization, vacuum drying and the like can be mentioned in that regard. The dry cell to be used in the present invention can be obtained by subjecting a cell of a microorganism to such treatments. The spray drying, ventilation drying, lyophilization, vacuum drying and the like can be performed under conditions generally known to those of ordinary skill in the art. In the spray drying, for example, the obtained cell is re-suspended at a cell concentration of about 5-25 wt % and dried at a temperature of 100-300° C. In the ventilation drying, for example, the obtained cell is applied in a wet state at a temperature of 100-700° C. In the lyophilization, for example, the obtained cell is re-suspended in water at a cell concentration of about 5-25 wt %, and the suspension is lyophilized. In vacuum drying, for example, the obtained cell is treated in a wet state at a temperature of 50-100° C. in vacuo (e.g., not more than 53 kPa). The above-mentioned conditions are mere examples and are not limitative. In spray drying, ventilation drying and the like, sterilization with heating may be or may not be performed before harvesting. In lyophilization and the like, however, sterilization with heating and the like are performed before harvesting so as to obtain a dead cell.

A dry cell of a microorganism for amino acid fermentation or nucleic acid fermentation can be used as it is as a deodorizing agent or can be formulated with one or more other components into a deodorizing preparation. Additives such as various sugars (e.g., lactose, glucose and the like), polysaccharides (e.g., starch, cellulose and the like), various proteins (e.g., casein and the like), various salts (e.g., calcium carbonate and the like), and the like may be present in the deodorizing preparation. The deodorizing agent or preparation may be in any form and, for example, powder, granule, tablet, capsule and the like can be mentioned in that regard. The deodorizing preparation can be produced by conventional means for formulating preparations.

The deodorizing agent or preparation can be used as it is or upon mixing with sawdust, zeolite, wood pieces, papers, activated carbon and the like.

As a deodorizing method of using the deodorizing agent of the present invention, the deodorizing agent and an odor substance are brought into contact with each other.

For example, when an odor substance is gaseous and fills a room and the like, the deodorizing agent may be placed in the room.

The amount of the thus prepared deodorizing agent based on the amount of a dry cell itself is preferably about 0.1 to 5 w/v %, when ammonia concentration per space volume is 500 ppm. With this amount of deodorizing agent, the ammonia in a room can be deodorized to an ammonia concentration that barely allows sensing by a person.

### EXAMPLES

The present invention is explained in more detail in the following Examples, which are not to be construed as limitative.

### Example 1

Preparation of Dry Cell from Lysine Fermentation Culture Medium

Lysine fermentation using L-lysine producing bacteria Escherichia coli FERM BP-5252 was performed in the following medium: Glucose 100 g/L, ammonium sulfate 60 g/L, KH₂PO₄ 1 g/L, MgSO₄.7H₂O 0.4 g/L, FeSO₄.7H₂O 10 mg/L, MnSO₄.4H₂O 8.1 mg/L, biotin 300 μg/L, thiamine hydrochloride 200 μg/L, soybean protein acid hydrolysate (total nitrogen content 3.2%) 35 ml/L, L-methionine 200 mg/L, calcium carbonate 50 g/L, pH 7.0.

The L-lysine producing bacterial strain was inoculated to the above-mentioned medium, and cultured at 36° C. for 72 hr. After completion of the lysine fermentation, the fermentation broth was sterilized with heating. The heat sterilization condition was 120° C., 2 minutes. Thereafter, the sterilization liquid was separated by centrifugation into a heavy liquid containing the cell and a light liquid. The obtained cell-containing liquid contains the cell in a proportion of about 4 to 20 wt %. This was further concentrated under reduced pressure to give a cell concentration liquid having a cell content of about 15 to 25 wt %. The obtained cell concentration liquid was spray dried at a hot wind temperature of 150 to 250° C. to give a dry cell having a water content of 2 to 10 wt %.

### Example 2

Ammonia Deodorizing Effect of Dry Cell of Lysine Fermentation Microorganism

The dry cell of lysine fermentation microorganism obtained in Example 1 was used. In 3L bags controlled to have an ammonia gas concentration of 500 ppm were independently placed an activated carbon powder (1 g) and a dry cell of lysine fermentation microorganism (dry lysine cell, 5 g), and the gas concentration in the bags was determined 2, 5, 10, 30 and 60 minutes later by a gas detector. The gas concentration in the bag without deodorizing treatment was also determined.

TABLE 1

| | Lapse of time (min.) | | | | |
|---|---|---|---|---|---|
| Sample | 2 | 5 | 10 | 30 | 60 |
| Dry lysine cell | 400 | 250 | 140 | 20 | <10 |
| Activated carbon powder | 200 | 170 | 140 | 80 | 60 |
| No addition | 500 | 500 | 500 | 480 | 440 |

(unit: ppm)

### Example 3

Deodorize Effect of Dry Cell of Lysine Fermentation Microorganism on Trimethylamine

The dry cell of lysine fermentation microorganism obtained in Example 1 was used. In 3L bags controlled to have a trimethylamine gas concentration of 50 ppm were independently placed an activated carbon powder (1 g), and a dry cell of lysine fermentation microorganism (dry lysine cell, 5 g), and the gas concentration in the bags was determined 2, 5, 10, 30 and 60 minutes later by a gas detector. The gas concentration in the bag without deodorizing treatment was also determined.

TABLE 2

| | Lapse off time (min.) | | | | |
|---|---|---|---|---|---|
| Sample | 2 | 5 | 10 | 30 | 60 |
| Dry lysine cell | 13 | 11 | 4 | <1 | Not measured |
| Activated carbon powder | 7 | 3 | 2 | 1 | <1 |
| No addition | 50 | 50 | 49 | 49 | 48 |

(unit: ppm)

### Example 4

Preparation of Dry Cell from Phenylalanine Fermentation Culture Medium

Phenylalanine fermentation using L-phenylalanine fermentation bacteria Brevibacterium lactofermentum FERM BP-4160 was performed in the following medium: Sucrose 2%, potassium phosphate 0.1%, magnesium sulfate 0.04%, ferrous sulfate 0.001%, manganese sulfate 0.01%, ammonium acetate 0.4%, soybean protein acid hydrolysate (as total nitrogen) 0.2%, L-tyrosine 0.04%, biotin 1000 μg/L and vitamin B₁ 100 μg/L.

The L-phenylalanine producing bacterial strain was inoculated to the above-mentioned medium, and the cells were cultured at 31° C. for 24 hr.

After the completion of the phenylalanine fermentation, a filter aid was added to the fermentation broth, and the mixture was thereafter separated by a compression type filtration separator into a filtration residue containing the cell and a light liquid. The obtained filtration residue contained the cells in an amount of about 20 to 50 wt %, which was aeration dried with hot air at a temperature of 150 to 600° C. to give a dry cell having a water content of 2 to 10 wt %.

### Example 5

Deodorizing Effect of Dry Cell of Phenylalanine Fermentation Microorganism on Ammonia

The dry cell of phenylalanine fermentation microorganism obtained in Example 4 was used. In 3L bags controlled to have an ammonia gas concentration of 500 ppm were independently placed an activated carbon powder (1 g), and a dry cell of phenylalanine fermentation microorganism (dry phenylalanine cell, 5 g), and the gas concentration in the bags was determined 2, 5, 10, 30 and 60 minutes later by a gas detector. The gas concentration in the bag without deodorizing treatment was also determined.

TABLE 3

| | Lapse of time (min.) | | | | |
|---|---|---|---|---|---|
| Sample | 2 | 5 | 10 | 30 | 60 |
| Dry phenylalanine cell | 90 | 60 | 30 | 10 | <10 |
| Activated carbon powder | 100 | 80 | 60 | 30 | 20 |
| No addition | 500 | 500 | 500 | 480 | 480 |

(unit: ppm)

### Example 6

Preparation of Dry Cell from Phenylalanine Fermentation Culture Medium

Phenylalanine fermentation using L-phenylalanine fermentation bacteria Bacillus subtilis FERM BP-609 was performed in the following medium: Glucose 2%, potassium phosphate 0.1%, magnesium sulfate 0.04%, ferrous sulfate 0.002%, manganese sulfate 0.002%, soybean protein acid hydrolysate (as total nitrogen) 0.2%, L-tyrosine 0.02%, L-tryptophan 0.02%.

The L-phenylalanine producing bacterial strain was inoculated to the above-mentioned medium, and the cells were cultured at 30° C. for 24 hr. After the completion of the phenylalanine fermentation, a filter aid was added to the fermentation broth, and the mixture was thereafter separated by a compression type filtration separator into a filtration residue containing the cell and a light liquid. The obtained filtration residue contained the cells in an amount of about 20 to 50 wt %, which was aeration dried with hot air at a temperature of 150 to 600° C. to give a dry cell having a water content of 2 to 10 wt %.

### Example 7

Deodorizing Effect of Dry Cell of Phenylalanine Fermentation Microorganism on Acetic Acid

The dry cell of phenylalanine fermentation microorganism obtained in Example 6 was used. In 3L bags controlled to have an acetic acid gas concentration of 50 ppm were independently placed an activated carbon powder (1 g), and a dry cell of phenylalanine fermentation microorganism (dry phenylalanine cell, 5 g), and the gas concentration in the bags was determined 2, 5, 10, 30 and 60 minutes later by a gas detector. The gas concentration in the bag without deodorizing treatment was also determined.

TABLE 4

| | Lapse of time (min.) | | | | |
|---|---|---|---|---|---|
| Sample | 2 | 5 | 10 | 30 | 60 |
| Dry phenylalanine | 6 | 5 | 3 | 2 | 1 |
| cell | | | | | |
| Activated carbon | 1 | <1 | Not | Not | Not |
| powder | | | measured | measured | measured |
| No addition | 51 | 50 | 50 | 50 | 40 |

(unit: ppm)

### Example 8

Comparison of Deodorizing Effect of Dry Cells of Lysine Fermentation Microorganism and Yeast on Ammonia

The dry cell of lysine fermentation microorganism obtained in Example 1 was used. As a control for comparison, a dry cell of yeast (dry beer yeast manufactured by KIRIN BREWERY CO., LTD.) was used. In 3L bags controlled to have an ammonia gas concentration of 500 ppm were independently placed an activated carbon powder (1 g), dry yeast cell (5 g) and a dry cell of lysine fermentation microorganism (dry phenylalanine cell, 5 g), and the gas concentration in the bags was determined 2, 5, 10, 30 and 60 minutes later by a gas detector. The gas concentration in the bag without deodorizing treatment was also determined.

TABLE 5

| | Lapse of time (min.) | | | | |
|---|---|---|---|---|---|
| Sample | 2 | 5 | 10 | 30 | 60 |
| Dry lysine cell | 20 | <10 | Not | Not | Not |
| | | | measured | measured | measured |
| Dry yeast cell | 120 | 100 | 40 | <10 | Not |
| | | | | | measured |
| Activated carbon | 200 | 170 | 140 | 80 | 60 |
| powder | | | | | |
| No addition | 500 | 500 | 500 | 480 | 440 |

(unit: ppm)

### Example 9

Deodorizing Effect of Dry Cell of Lysine Fermentation Microorganism on Ammonia in Broiler Dropping

The dry cell of lysine fermentation microorganism obtained in Example 1 was used. Broiler litter (chaff) (250 g) was mixed with 1% lysine fermentation cell and 3% of zeolite (commercially available), respectively, and the mixture was mixed well with 750 g of broiler dropping. The gas concentration was determined 4, 6, 8 and 10 days later by a gas detector. The gas concentration in the bag without deodorizing treatment was also determined.

TABLE 6

Lapse of time (min.)

| Sample | 4 | 6 | 8 | 10 |
|---|---|---|---|---|
| Dry lysine cell | 5 | 17 | 130 | 260 |
| Zeolite | 11 | 43 | 189 | 305 |
| No addition | 5 | 60 | 254 | 396 |

(unit: ppm)

From the above-mentioned Examples 2, 3, 5, 7, 8 and 9, it has been confirmed that, by contacting amino acid fermentation or nucleic acid fermentation cells with an offensive odor substance, the concentration of the offensive odor substance was reduced, thus demonstrating a deodorizing effect.

While the reason for the deodorizing effect of a dried cell of a particular bacterium on an offensive odor substance is not clear, it is postulated that a dried cell surface is charged by drying the cell, and then becomes electrically bonded to the charged offensive odor substance. On the other hand, cells are aggregated by drying, forming micropores in the cake (lump), as a result of which a deodorizing effect is achieved based on physical adsorption, like activated carbon and the like. Consequently, the dried cell electrically and physically adsorbs the offensive odor substance, and therefore, the deodorizing agent of the present invention exhibits a remarkable effect as compared to activated carbon.

From the above results, the deodorizing effect is considered to be achieved by drying an amino acid or nucleic acid fermentation cell irrespective of the drying method.

According to the deodorizing agent and the deodorizing method of the present invention, particularly an offensive odor caused by excreta etc. produced during the breeding of domestic animals and indoor breeding of pet animals such as dogs, cats, and the like can be effectively deodorized. Since the amino acid and nucleic acid fermentation cells after deodorizing are organic materials, they can be beneficially burned or reused as a fertilizer and the like.

While some of the embodiments of the present invention have been described in detail in the above, it will, however, be evident for those of ordinary skill in the art that various modifications and changes may be made to the particular embodiments shown without substantially departing from the novel teaching and advantages of the present invention. Such modifications and changes are encompassed in the spirit and scope of the present invention as set forth in the appended claims.

This application is based on a patent application No. 004839/2004 filed on Jan. 9, 2004 in Japan, the contents of which are hereby incorporated by reference.

1. A deodorizing agent comprising, as an active ingredient, a dry cell of a microorganism selected from the group consisting of bacteria belonging to the genus Escherichia, bacteria belonging to the genus Brevibacterium, and bacteria belonging to the genus Bacillus.

2. The deodorizing agent of claim 1, wherein the microorganism is a bacterium belonging to the genus Escherichia.

3. The deodorizing agent of claim 1, wherein the microorganism is a bacterium belonging to the genus Brevibacterium.

4. The deodorizing agent of claim 1, wherein the microorganism is a bacterium belonging to the genus Bacillus.

5. A deodorizing method comprising contacting an odor substance with a deodorizing agent comprising, as an active ingredient, a dry cell of a microorganism selected from the group consisting of bacteria belonging to the genus Escherichia, bacteria belonging to the genus Brevibacterium, and bacteria belonging to the genus Bacillus.

6. The deodorizing agent of claim 5, wherein the microorganism is a bacterium belonging to the genus Escherichia.

7. The deodorizing method of claim 6, wherein the odor substance is at least one kind of odor substance selected from the group consisting of ammonia, trimethylamine, methyl ercaptan, hydrogen sulfide, and fatty acid

8. The deodorizing method of claim 7, wherein the fatty acids are propionic acid and n-butyric acid.

9. The deodorizing agent of claim 5, wherein the microorganism is a bacterium belonging to the genus Brevibacterium.

10. The deodorizing method of claim 9, wherein the odor substance is at least one kind of odor substance selected from the group consisting of ammonia, trimethylamine, methyl mercaptan, hydrogen sulfide, and fatty acid

11. The deodorizing method of claim 10, wherein the fatty acids are propionic acid and n-butyric acid.

12. The deodorizing agent of claim 5, wherein the microorganism is a bacterium belonging to the genus Bacillus.

13. The deodorizing method of claim 12, wherein the odor substance is at least one kind of odor substance selected from the group consisting of ammonia, trimethylamine, methyl mercaptan, hydrogen sulfide, and fatty acid

14. The deodorizing method of claim 13, wherein the fatty acids are propionic acid and n-butyric acid.
